(19) United States
(12) Patent Application Publication
(10) Pub. No.: US 2017/0343367 A1
(43) Pub. Date: Nov. 30, 2017

(54) APPARATUS AND METHOD FOR ANTICIPATING AND FACILITATING VEHICLE OCCUPANT'S METABOLIC ACTIVITY

(71) Applicant: HYUNDAI MOTOR COMPANY, Seoul (KR)

(72) Inventors: Hwal Ly LEE, Seoul (KR); Jin Kwon KIM, Suwon-si (KR)

(21) Appl. No.: 15/255,924

(22) Filed: Sep. 2, 2016

(30) Foreign Application Priority Data
May 26, 2016 (KR) 10-2016-0064960

(51) Int. Cl. G01C 21/34; G01C 21/36; A61B 5/20; A61B 5/00; H04W 64/00; H04L 29/08; H04W 84/04; H04W 88/02

(52) U.S. Cl. CPC G01C 21/3484; A61B 5/6893; G01C 21/3617; H04W 64/006; A61B 5/204; G01C 21/3679; H04W 84/042; H04W 88/02; H04L 67/12

(57) ABSTRACT

An apparatus for anticipating and facilitating metabolic activity of an occupant in a vehicle includes a measurement part measuring a volume of a bladder using a sensor mounted in the vehicle; a rest area information calculator calculating information on a rest area including a distance to the rest area which may be reached within a predetermined time or an expected time of arrival to the rest area which may be reached, according to rest area location information and vehicle speed information; and a guidance part providing indication information including the volume of the bladder or the information on the rest area.

### CROSS-REFERENCE TO RELATED APPLICATION

This application claims the benefit of priority to Korean Patent Application No. 10-2016-0064960, filed on May 26, 2016 in the Korean Intellectual Property Office, the entirety of which is incorporated herein by reference.

### TECHNICAL FIELD

The present disclosure relates to an apparatus and a method for anticipating and facilitating a passenger's metabolic activity, and more particularly, to an apparatus and a method for sensing a passenger's metabolic activity and determining whether or not the possibility of guiding an occupant such as a driver or a passenger to an appropriate facility in advance and automatically providing such guidance information in advance.

### BACKGROUND

When driving a long distance or driving on a road in which toilets are rare, in particular, drivers accompanied by a baby or a young child sometimes experience embarrassment due to unexpected metabolic activity. Furthermore, not only children, but many adult drivers also experience embarrassment because of metabolic activity but may pass rest areas during holidays or on a congested highway.

In regard to such embarrassing metabolic activity, there is a system providing an alarm to a guardian or a user when humidity is increased by attaching a humidity measurement sensor to clothes of children or a critical patient, or a bed sheet. In addition, a smart diaper technology, or the like, is known where a QR code or a label is attached to a diaper or the bed sheet, such that a urine analysis is performed by a scanning of a portable device, and nutritive condition, bacteria and disease of a subject may be analyzed.

However, such metabolism-related technology corresponds to a healthcare technology that performs a post-processing or analyzes a health state after the metabolic activity has already occurred, and does not provide guidance in advance before the metabolic activity occurs. Therefore, there has been research conducted into a technology capable of sensing and predicting the metabolic activity in advance at the time of driving a long distance or driving on a congested road.

### SUMMARY

The present disclosure has been made to solve the above-mentioned problems occurring in the prior art while advantages achieved by the prior art are maintained intact.

An aspect of the present disclosure provides an apparatus and a method for guiding a driver or passenger to a convenient facility, and is capable of automatically inducing a visit to such a facility or providing a guidance service in advance by sensing metabolic activity such as whether or not the possibility of arriving at the facility in advance using a sensor mounted in a vehicle.

According to an exemplary embodiment of the present disclosure, an apparatus for anticipating and facilitating metabolic activity of an occupant in a vehicle includes: a measurement part measuring a volume of a bladder using a sensor mounted in the vehicle; a rest area information calculator calculating information on a rest area including a distance to the rest area which may be reached within a predetermined time or an expected time of arrival to the rest area which may be reached, from rest area location information and vehicle speed information; and a guidance part providing indication information including the volume of the bladder or the information on the rest area.

The guidance part may provide the indication information to a display device included in the vehicle, or provide the indication information to a user terminal which is interworked in a local area network or a mobile communication network.

The guidance part may further provide a pre-induction notification message or an alarm sound inducing a visit to the rest area which may be reached within the predetermined time as the indication information, in addition to information on the volume of the bladder or the information on the rest area, when the volume of the bladder is a threshold volume or more.

The apparatus may further include a starting controller generating a starting signal when a button is turned on, or repetitively generating the starting signal at a period after the button is turned on. The apparatus may further include a starting controller generating the starting signal when the distance to the rest area which is able to arrive within the predetermined time is a threshold distance or less, or the expected time of arrival is a threshold time or less, on the basis of the information on the rest area.

The measurement part may measure the volume of the bladder of the occupant by controlling ultrasonic wave occurrences from a plurality of ultrasonic sensors, and measuring an incident wave and a reflected wave of an ultrasonic wave at each of the plurality of ultrasonic sensors.

The rest area information calculator may receive global positioning system (GPS) information as the rest area location information from a navigation device, and receive the vehicle speed information through a vehicle sensor or a control area network (CAN) bus.

According to another exemplary embodiment in the present disclosure, a method for anticipating and facilitating metabolic activity of an occupant in a vehicle includes: measuring a volume of a bladder using a sensor mounted in the vehicle; calculating information on a rest area including a distance to the rest area which may be reached within a predetermined time or an expected time of arrival to the rest area which may be reached, from rest area location information and vehicle speed information; and providing indication information including the volume of the bladder or the information on the rest area.

In the providing of the indication information, the indication information may be provided to a display device included in the vehicle, or the indication information may be provided to a user terminal which is interworked in a local area network or a mobile communication network.

In the providing of the indication information, a pre-induction notification message or an alarm sound inducing a visit to the rest area which may be reached within the predetermined time may be further provided as the indication information, in addition to information on the volume of the bladder or the information on the rest area, when the volume of the bladder is a threshold volume or more.

The method may further include generating a starting signal when a button is turned on, or repetitively generating the starting signal at a period after the button is turned on.

The method may further include generating a starting signal when the distance to the rest area which may be reached within the predetermined time is a threshold distance or less, or the expected time of arrival is a threshold time or less, on the basis of the information on the rest area.

In the measuring of the volume of the bladder, the volume of the bladder of the occupant may be measured by controlling ultrasonic wave occurrences from a plurality of ultrasonic sensors, and measuring an incident wave and a reflected wave of an ultrasonic wave at each of the plurality of ultrasonic sensors.

In the calculating of the information on the rest area, global positioning system (GPS) information may be received as the rest area location information from a navigation device, and the vehicle speed information may be received through a vehicle sensor or a control area network (CAN) bus.

### BRIEF DESCRIPTION OF THE DRAWINGS

The above and other objects, features and advantages of the present disclosure will be more apparent from the following detailed description taken in conjunction with the accompanying drawings.

FIG. 1 is a diagram illustrating an apparatus for anticipating and facilitating metabolic activity according to an exemplary embodiment in the present disclosure.

FIG. 2 is a flowchart illustrating an operation of the apparatus for anticipating and facilitating metabolic activity according to an exemplary embodiment in the present disclosure.

FIG. 3 is a diagram illustrating an installation example of a sensor for measuring a volume of a bladder according to an exemplary embodiment in the present disclosure.

FIG. 4 is a diagram illustrating a volume measurement of the bladder using an ultrasonic sensor for measuring a volume of a bladder according to an exemplary embodiment in the present disclosure.

FIG. 5 is an example of a screen of a user terminal illustrating examples of display information and a pre-induction alarm service according to an exemplary embodiment in the present disclosure.

FIG. 6 is a diagram illustrating an example of an implementation method of an apparatus for anticipating and facilitating metabolic activity according to an exemplary embodiment in the present disclosure.

### DETAILED DESCRIPTION

Hereinafter, the present disclosure will be described in detail with reference to the accompanying drawings. Here, like reference numerals denote like elements in the respective drawings. In addition, a detailed description of functions and/or configurations which are already known will be omitted. The contents disclosed below mainly describe portions necessary to understand operations according to various exemplary embodiments and a description of elements which may obscure the gist of the description will be omitted. In addition, some components shown in the drawings may be exaggerated, omitted or schematically illustrated. The size of each component does not exactly reflect its real size and accordingly, the contents described in this specification are not limited by relative sizes or intervals of the components illustrated in the respective drawings.

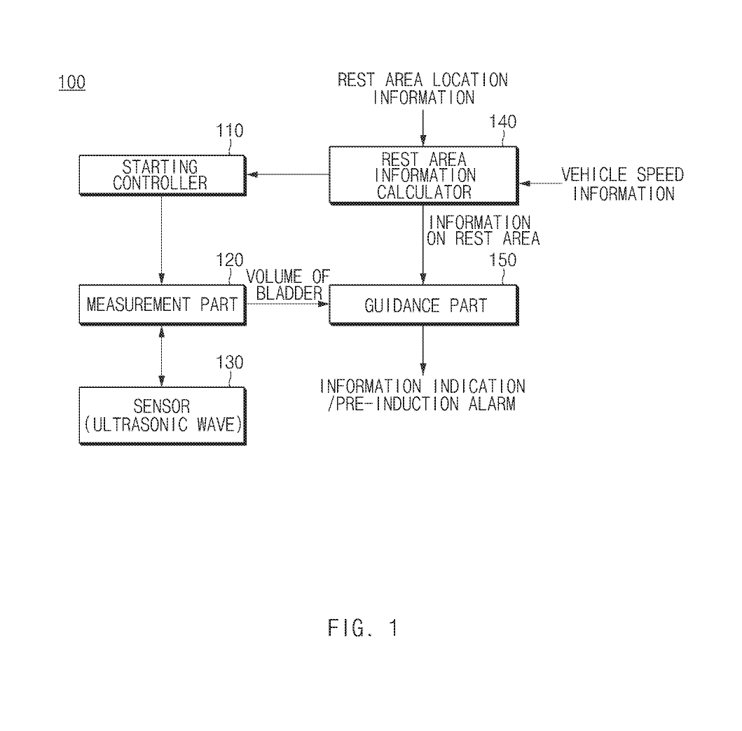

FIG. 1 is a diagram illustrating an apparatus 100 for anticipating and facilitating metabolic activity according to an exemplary embodiment in the present disclosure.

Referring to FIG. 1, the apparatus 100 for anticipating and facilitating metabolic activity according to an exemplary embodiment includes a starting controller 110, a measurement part 120, a sensor 130, a rest area information calculator 140, and a guidance part 150. The respective components of the apparatus 100 for anticipating and facilitating metabolic activity according to an exemplary embodiment as described above may be implemented in hardware such as a semiconductor processor, software such as an application program, or a combination thereof.

First, an operation of the apparatus 100 for anticipating and facilitating metabolic activity according to an exemplary embodiment will be briefly described.

The starting controller 110 may generate a starting signal for anticipating and facilitating an occupant's metabolic activity when a button is turned on, or may repetitively generate the starting signal for anticipating and facilitating the occupant's metabolic activity at a period after the button is turned on. Such button may be installed at a proper position such as a steering wheel, a center console, a door trim, or the like so that a driver may easily manipulate the button from a driver's seat. Besides, in the case in which a distance to a rest area which may be reached within a predetermined time (e.g., within 1 hour) is a threshold distance or less, or an expected time of arrival to the corresponding rest area is a threshold time or less, on the basis of rest area information calculated by the rest area information calculator 140 as described below, the starting controller 110 may also generate the starting signal for anticipating and facilitating the occupant's metabolic activity.

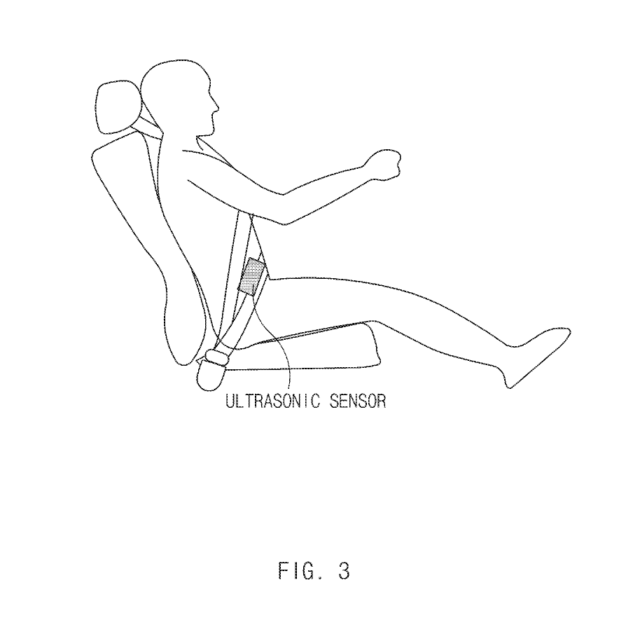

The measurement part 120 measures a volume of a bladder using a sensor mounted in the vehicle according to the above-mentioned starting signal. For example, a plurality of ultrasonic sensors 130 (see FIG. 3) may be installed at a proper position such as a seat belt or a seat of the vehicle. As described below, the measurement part 120 may measure the volume of the bladder of an occupant by controlling ultrasonic wave occurrences from the plurality of ultrasonic sensors and measuring an incident wave and a reflected wave of an ultrasonic wave at each of the plurality of ultrasonic sensors.

The rest area information calculator 140 calculates information on the rest area including the distance to the rest area which is able to arrive within the predetermined time (e.g., within 1 hour) or the expected time of arrival to the corresponding reachable rest area from global positioning system (GPS) based rest area location information and vehicle speed information (e.g., average vehicle speed information per hour). For example, the rest area information calculator 140 may receive GPS information (e.g., information such as latitude, longitude, altitude, and the like) as the rest area location information from a navigation device (or a terminal) performing a predetermined navigation algorithm, and may receive the vehicle speed information through a vehicle sensor or a control area network (CAN) bus.

The guidance part 150 provides indication information (e.g., display information, alarm warning information, or the like) including the volume of the bladder measured as described above or the information on the rest area calculated by the rest area information calculator 140. The guidance part 150 may also provide the above-mentioned indication information to a display device (e.g., a navigation, a cluster, or the like) included in the vehicle, or may also provide the indication information to a user terminal which is interworked in a local area network (e.g., Zigbee, Bluetooth, or the like) or a mobile communication network (e.g., (W)CDMA, WiFi, LTE, WiBro, or the like). The user terminal, which is a terminal carried by the user such as a driver, an occupant, a passenger, or the like, includes a wireless terminal such as a smartphone, a wearable device capable of voice/video call, a tablet PC, a notebook PC, or the like.

In the case in which the volume of the bladder measured as described above is a threshold volume or more, the guidance part 150 may further provide indication information such as a pre-induction notification message (e.g., a character display “please visit the rest area”) inducing the visit to the above-mentioned rest area which may be reached within the predetermined time (e.g., within 1 hour) or an alarm sound (e.g., a voice message “please visit the rest area”) to the display device (e.g., a navigation, a cluster, or the like) included in the vehicle, the user terminal, or the like, in addition to information (e.g., a measurement of 450a) on the volume of the bladder or the information on the rest area (e.g., a distance or a time to a next rest area) calculated by the rest area information calculator 140.

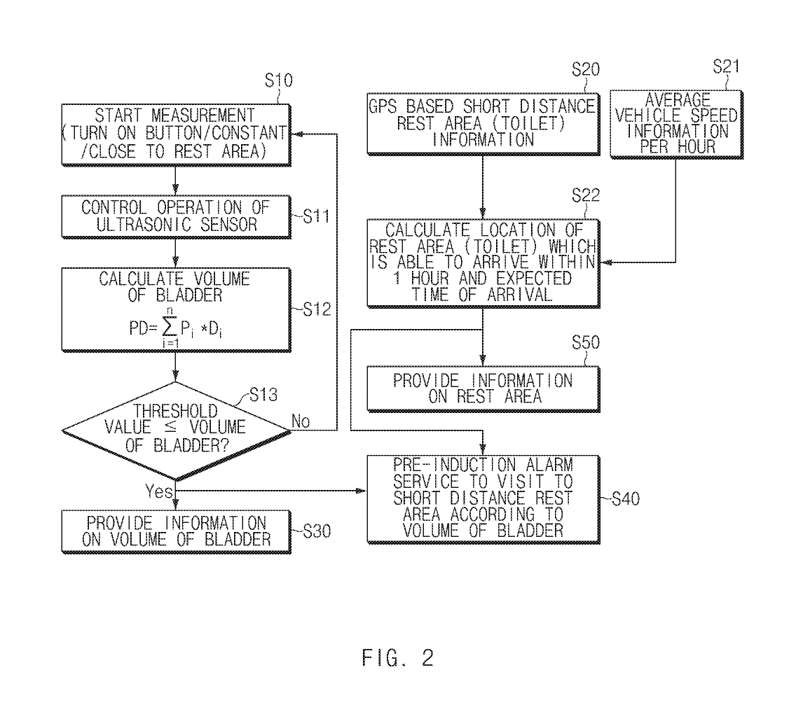

FIG. 2 is a flowchart illustrating an operation of the apparatus 100 for guiding metabolism according to an exemplary embodiment in the present disclosure.

Referring to FIG. 2, first, in order to start a metabolic guidance for a passenger such as a driver, an occupant, or the like, a starting signal is generated (S10). The driver may turn on a button installed at a proper position such as a steering wheel, a center console, a door trim, or the like, and in this case, the starting controller 110 may generate the starting signal. Besides, the starting controller 110 may also repetitively generate the starting signal at a period after the above-mentioned button is turned on. Besides, in the case in which a distance to a rest area which may be reached within a predetermined time (e.g., within 1 hour) is a threshold distance or less, or an expected time of arrival to the corresponding rest area is a threshold time or less, on the basis of information on the rest area (e.g., a distance or time to a next rest area) which may be reached within the predetermined time (e.g., within 1 hour) calculated by the rest area information calculator 140, the starting controller 110 may also generate the starting signal for anticipating and facilitating the occupant's metabolic activity.

As such, if the starting signal is generated, the measurement part 120 controls an operation of a sensor mounted in a vehicle (S11) to measure a volume of a bladder (S12). For example, a plurality of ultrasonic sensors 130 may be installed at a proper position such as a seat belt or a seat of the vehicle (see FIG. 3), and the measurement part 120 may measure the volume of the bladder of the occupant by controlling ultrasonic wave occurrences from the plurality of ultrasonic sensors and measuring an incident wave and a reflected wave of an ultrasonic wave at each of the plurality of ultrasonic sensors.

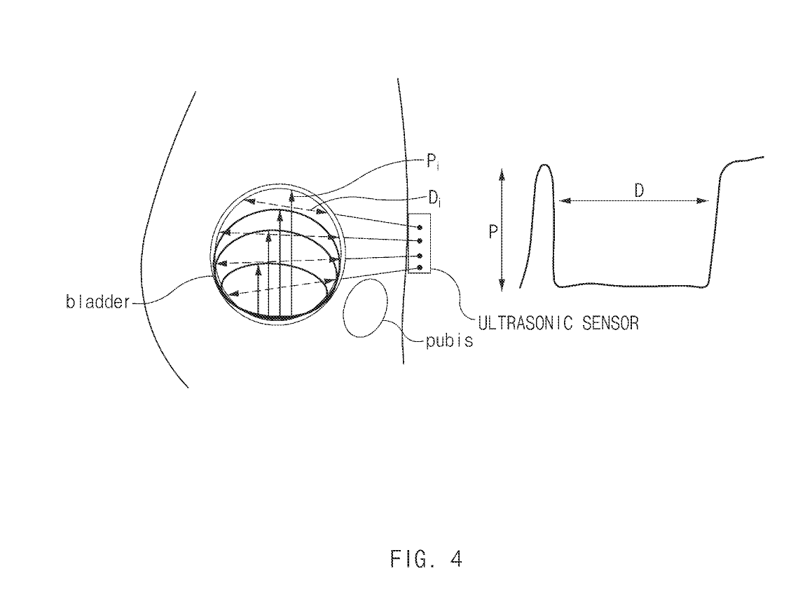

For example, as illustrated in FIG. 4, the plurality of ultrasonic sensors 130 may be positioned around the bladder when the occupant takes a seat belt or a seat of the vehicle. The bladder is anatomically positioned behind a pubis of a pelvis, and has a structure which is expanded and contracted while discharging urine through a urethra. A bottom of the bladder maintains a shape thereof even in the case in which the bladder is expanded and contracted, and an empty bladder is contracted in a state in which an upper portion of the bladder is flat, so as to be flatly attached to the bottom. A shape of the bladder as described above is a shape similar to a bendable disk.

The volume PD of the bladder may be calculated as a summation of Pᵢ*Dᵢ, when the number of ultrasonic beams of the ultrasonic sensors 130 is n, as expressed in Equation 1.

$$PD = {\sum }_{i = 1}^{n} {P}_{i} * {D}_{i} \qquad [ Equation 1 ]$$

Here, Pᵢ is echo intensities (dB) of reflected waves that incident ultrasonic waves are reflected from a back wall of the bladder (a wall opposite to a side at which the ultrasonic waves are positioned), and Dᵢ is a distance calculated by a time difference between the respective reflected waves reflected from the back wall and a front wall of the bladder. Here, Pᵢ is a variable reflecting a width (horizontal width) of the bladder, Dᵢ is a variable reflecting a depth (front and back length) of the bladder, and the number n of ultrasonic wave beams is a variable reflecting a height (vertical length) of the bladder. Since a method for calculating a volume of a bladder as described above is well known, a more detailed description will be omitted. Here, the number n of ultrasonic wave beams is generally four, but may be added or subtracted depending on a measurement situation such as noise, or the like according to a driving situation.

If the measured volume PD of the bladder is a threshold volume or more (S13), the guidance part 150 may provide indication information (e.g., display information, alarm warning information, or the like) including the volume of the bladder measured as described above to a display device (e.g., a navigation, a cluster, or the like) included in the vehicle or a user terminal (e.g., a smartphone, a wearable device, or the like) (S30). However, even in the case in which the volume PD of the bladder is smaller than the threshold volume, the above-mentioned indication information may also be provided to the display device (e.g., the navigation, the cluster, or the like) included in the vehicle or the user terminal (e.g., the smartphone, the wearable device, or the like). Alternatively, in the case in which the volume PD of the bladder is smaller than the threshold volume and in the case in which the volume PD of the bladder is the threshold volume or more, it is also possible to differently indicate the indication message or a warning sound.

Here, the threshold volume may be preset to 400 to 500 ml which may be expected for a normal adult based on medical information, and may be preset to 200 to 250 ml, which is about a half of the threshold volume of the adult, with respect to a baby or a young child in relation to the threshold volume of the adult. However, a consumer such as the driver, or the like may also set a value for the threshold volume by properly inputting the value for the threshold volume through the guidance part 150 or a user interface.

The rest area information calculator 140 calculates information on a rest area including the distance to the rest area which may be reached within the predetermined time (e.g., within 1 hour) or the expected time of arrival to the corresponding reachable rest area (S22) from global positioning system (GPS) based rest area location information (S20) and vehicle speed information (e.g., average vehicle speed information per hour) (S21). For example, the rest area information calculator 140 may receive GPS information (e.g., information such as latitude, longitude, altitude, and the like) as the rest area location information from a navigation device (or a terminal) performing a predetermined navigation algorithm (S20), and may receive the vehicle speed information through a vehicle sensor or a control area network (CAN) bus (S21).

The guidance part 150 may provide the indication information (e.g., the display information, the alarm warning information, or the like) including the information on the rest area such as the distance to the rest area which may be reached within the predetermined time (e.g., within 1 hour), the expected time of arrival to the corresponding rest area, or the like calculated by the rest area information calculator 140 as described above to the display device (e.g., the navigation, the cluster, or the like) included in the vehicle or the user terminal (e.g., the smartphone, the wearable device, or the like) (S50).

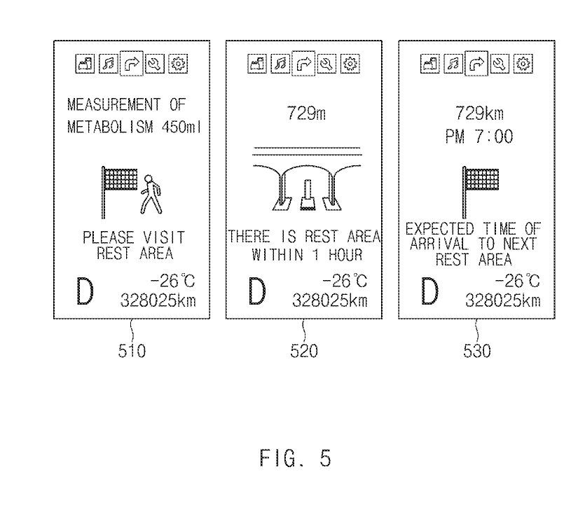

Besides, in the case in which the volume of the bladder measured as described above is the threshold volume or more, the guidance part 150 may further provide indication information such as a pre-induction notification message (e.g., a character display “please visit the rest area”) or an alarm sound (e.g., a voice message “please visit the rest area”) inducing the visit to the above-mentioned rest area which may be reached within the predetermined time (e.g., within 1 hour) to the display device (e.g., a navigation, a cluster, or the like) included in the vehicle, the user terminal, or the like as in the operation 510 of FIG. 5, in addition to the information (e.g., see the operation 510 of FIG. 5) on the volume of the bladder or the information on the rest area (e.g., see the operations 520 and 530 of FIG. 5) calculated by the rest area information calculator 140. A pre-visit of the driver and a child passenger to the rest area is induced through the above-mentioned guidance message or alarm, thereby making it possible to avoid an embarrassing situation which may occur in the middle of a path, and to provide accommodation.

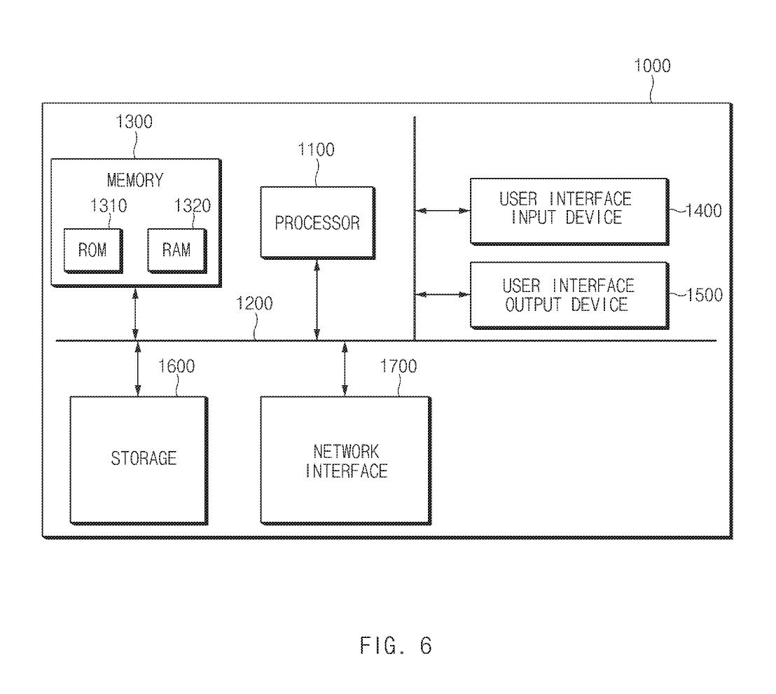

FIG. 6 is a diagram illustrating an example of an implementation method of an apparatus 100 for anticipating and facilitating metabolic activity according to an exemplary embodiment in the present disclosure. The apparatus 100 may be implemented in hardware, software, or a combination thereof. For example, the apparatus 100 for anticipating and facilitating metabolic activity may be implemented in a computing system 1000 as illustrated in FIG. 6.

The computing system 1000 may include at least one processor 1100, a memory 1300, a user interface input device 1400, a user interface output device 1500, a storage 1600, and a network interface 1700 which are connected through a bus 1200. The processor 1100 may be a central processing unit (CPU) or a semiconductor device executing processes for instructions which are stored in the memory 1300 and/or the storage 1600. The memory 1300 and the storage 1600 may include various kinds of volatile or non-volatile storing media. For example, the memory 1300 may include a read only memory (ROM) 1310 and a random access memory (RAM) 1320.

Accordingly, steps in the method or algorithm which is described in context with the exemplary embodiments disclosed in the present specification may be directly implemented in hardware, a software module, or a combination thereof which is executed by the processor 1100. The software module may be resided on a storing medium (i.e., the memory 1300 and/or the storage 1600) such as a RAM memory, a flash memory, a ROM memory, an erasable programmable read only memory (EPROM), an electrically erasable programmable read only memory (EEPROM), a register, a hard disk, a removable disk, or a compact disc-read only memory (CD-ROM). An exemplary storing medium may be coupled to the processor 1100 and the processor 1100 may read information from the storing medium and write the information into the storing medium. Alternatively, the storing medium may be integral with the processor 1100. The processor and the storing medium may also be resided within an application specific integrated circuit (ASIC). The ASIC may also be resided within a user terminal. Alternatively, the processor and the storing medium may also be resided within the user terminal as a separate component.

As described above, according to the exemplary embodiments in the present disclosure, the apparatus and the method for anticipating and facilitating metabolic activity automatically induces a visit to the rest area or the toilet or provides a guidance service in advance by sensing metabolic activity such as whether or not the possibility of evacuation when there is such a need for evacuation is provided to the driver in advance, thereby making it possible to increase the convenience of the customer.

Further, the apparatus and the method for anticipating and facilitating metabolic activity according to the present disclosure may provide a healthcare effect in an inexpensive form with low material cost and being mass-producible, thereby making it possible to secure marketability of the vehicle and provide a selling point. Further, the present apparatus and method may prevent a stop on a shoulder of the highway or reckless driving caused by unexpected situations associated with the metabolic activity which may occur during the driving, thereby making it possible to ultimately lead to a safer driving culture.

In addition, the apparatus and the method for anticipating and facilitating metabolic activity according to the present disclosure may further incorporate a technology related to determining problems in organs, or the like in the future, in addition to the volume of the bladder, so it is possible to expand and develop an additional new technology.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims.

What is claimed is:

1. An apparatus for anticipating and facilitating metabolic activity of an occupant in a vehicle, the apparatus comprising:
a measurement part measuring a volume of a bladder using a sensor mounted in the vehicle;
a rest area information calculator calculating information on a rest area including a distance to the rest area which may be reached within a predetermined time or an expected time of arrival to the rest area which may be reached, according to rest area location information and vehicle speed information; and
a guidance part providing indication information including the volume of the bladder or the information on the rest area.

2. The apparatus according to claim 1, wherein the guidance part provides the indication information to a display device included in the vehicle, or provides the indication information to a user terminal which is interworked in a local area network or a mobile communication network.

3. The apparatus according to claim 1, wherein the guidance part further provides a pre-induction notification message or an alarm sound inducing a visit to the rest area which may be reached within the predetermined time as the indication information, in addition to information on the volume of the bladder or the information on the rest area, when the volume of the bladder is a threshold volume or more.

4. The apparatus according to claim 1, further comprising a starting controller generating a starting signal when a button is turned on, or repetitively generating the starting signal at a period after the button is turned on.

5. The apparatus according to claim 1, further comprising a starting controller generating a starting signal when the distance to the rest area which may be reached within the predetermined time is a threshold distance or less, or the expected time of arrival is a threshold time or less, on the basis of the information on the rest area.

6. The apparatus according to claim 1, wherein the measurement part measures the volume of the bladder of the occupant by controlling ultrasonic wave occurrences from a plurality of ultrasonic sensors, and measuring an incident wave and a reflected wave of an ultrasonic wave at each of the plurality of ultrasonic sensors.

7. The apparatus according to claim 1, wherein the rest area information calculator receives global positioning system (GPS) information as the rest area location information from a navigation device, and receives the vehicle speed information through a vehicle sensor or a control area network (CAN) bus.

8. A method for anticipating and facilitating metabolic activity of an occupant in a vehicle, the method comprising steps of:
measuring a volume of a bladder using a sensor mounted in the vehicle;
calculating information on a rest area including a distance to the rest area which may be reached within a predetermined time or an expected time of arrival to the rest area which may be reached, from rest area location information and vehicle speed information; and
providing indication information including the volume of the bladder or the information on the rest area.

9. The method according to claim 8, wherein in the step of providing the indication information, the indication information is provided to a display device included in the vehicle, or the indication information is provided to a user terminal which is interworked in a local area network or a mobile communication network.

10. The method according to claim 8, wherein in the step of providing the indication information, a pre-induction notification message or an alarm sound inducing a visit to the rest area which may be reached within the predetermined time is further provided as the indication information, in addition to information on the volume of the bladder or the information on the rest area, when the volume of the bladder is a threshold volume or more.

11. The method according to claim 8, further comprising a step of generating a starting signal when a button is turned on, or repetitively generating the starting signal at a period after the button is turned on.

12. The method according to claim 8, further comprising a step of generating a starting signal when the distance to the rest area which may be reached within the predetermined time is a threshold distance or less, or the expected time of arrival is a threshold time or less, on the basis of the information on the rest area.

13. The method according to claim 8, wherein in the step of measuring the volume of the bladder, the volume of the bladder of the occupant is measured by controlling ultrasonic wave occurrences from a plurality of ultrasonic sensors, and measuring an incident wave and a reflected wave of an ultrasonic wave at each of the plurality of ultrasonic sensors.

14. The method according to claim 8, wherein in the step of calculating the information on the rest area, global positioning system (GPS) information is received as the rest area location information from a navigation device, and the vehicle speed information is received through a vehicle sensor or a control area network (CAN) bus.
